(19) United States
(12) Patent Application Publication
(10) Pub. No.: US 2011/0135772 A1
(43) Pub. Date: Jun. 9, 2011

(54) SKIN CARE AGENT AND COMPOSITIONS THEREOF

(75) Inventors: Gottumukkala Venkateswara Rao, Tamil Nadu (IN); Triptikumar Mukhopadhyay, Tamil Nadu (IN); Guruswamy Damodaran Rajesh, Tamil Nadu (IN)

(73) Assignee: CAVINKARE PVT. LTD., Tamil Nadu (IN)

(21) Appl. No.: 13/054,839

(22) PCT Filed: Mar. 16, 2009
(86) PCT No.: PCT/IN09/00181

§ 371 Date: Jan. 19, 2011

(51) Int. Cl. A61K 8/60; A61K 8/97; A61Q 19/00; A61Q 19/08

(52) U.S. Cl. 424/769; 514/25

(57) ABSTRACT

A skin care agent and cosmetic and/or dermopharmaceutical compositions involving selectively polygallate molecule either singly or as a synergistic mixture of a polygallate molecule and a gallic acid ester in an effective amount along with an acceptable vehicle with or without other skin benefit agents. Preferably, the active mixture or ingredients is selectively obtained by appropriate combination of suitable sources including plants rich in individual ingredients or a single source such as the skin and/or kernel of Mangifera indica Linn.

### FIELD OF THE INVENTION

The present invention relates to a skin care agent and cosmetic and/or dermopharmaceutical compositions obtained thereof and, in particular, to an anti-aging benefit agent for skin care and cosmetic and/or dermopharmaceutical compositions. Advantageously, the anti-aging benefit agent of the invention is preferably selectively obtained by appropriate combination of suitable sources including plants rich in individual ingredients or a single source such as selective extracts and/or active ingredients derived from skin and/or kernel of mango (fruit of Mangifera indica Linn.,). The invention further relates to processes to isolate the said selective extracts and/or active ingredients and to methods for making and using the compositions. Importantly, the present invention also relates more specifically to skin care compositions comprising selective extracts and/or active ingredients derived from skin and/or kernel of mango (fruit of Mangifera indica Linn.,) having preventive and/or therapeutic efficacy as a skin care composition and more importantly and surprisingly providing anti-aging attribute/effect when incorporated in cosmetically/dermopharmaceutically acceptable base/vehicle.

### BACKGROUND ART

Cosmetic preparations in various combinations are available and well known for use by intending consumers to cater to specific skin care requirements. The consumers now have growing consciousness and expectations from cosmetic preparation that should not only demonstrate certain care effects or overcome certain deficiency, but more often they demand for products, which are capable to demonstrate several benefit attributes simultaneously, and thus would exhibit an improved performance spectrum. Consumers of cosmetic products are particularly interested to obtain such multiple properties in the product that would on one hand provide care and vitalizing effect such as to obtain protection against aging phenomena for skin—both biological and actinic, skin lightening capabilities and also antioxidant property. A separate expectation of the consumers relate to the aspect of good skin compatibility and particularly the use of natural products in the preparation/formulation. More importantly, it is desirable to provide such skin care formulations from renewable sources such as plants, plant parts, fruits and seeds.

However, to provide for several benefit attributes it is usually found to be a requirement to combine multiple active ingredients which not only add to the complexity of the formulation but also affect the other related product attributes apart from being cost-extensive to obtain. There is, therefore, a continuing need to better the skin care attributes in skin care formulations and multiply the benefit attributes from less number of available sources of actives for such purposes thereby making the skin formulation more user friendly and cost effective.

The normal skin condition and appearance is a reflection of ones health and youthfulness. The skin condition may be altered by the effects of natural environmental factors, such as sunlight, heat/cold, humidity etc., or man made factors like indoor heating, air-conditioning, pollution, irritants/allergens, smoking, facial expressions etc., or pathological processes such as dermatological diseases or the normal aging process or even some medical treatments. The various damages to which the skin is exposed may act individually or synergistically and may be transient or permanent in nature. Visible aging of the skin starts at about age 25 as the natural regenerative process begin to slow. The skin replaces old cells at a lower rate including those at the skin surface. When skin is exposed to ultraviolet rays or other causative factors or simply ages, there is a reduction in level of mucopolysaccharides including reduction of the collagen or an elastin and hyaluronic acid level. An elastin forms bridge structures giving rise to a network, holds individual skin cells in place and contributes to the elasticity and firmness of the skin. When an elastin is denaturalized and destroyed, due to action of an elastase, which is an enzyme that destroys elastin and which is triggered by exposure to ultraviolet-rays or aging, there is break down of elastin bridge network leading to a reduction in the resiliency/elasticity of the skin. Therefore, for the prevention of the aging of the skin, it is important to suppress the functioning of elastase and thereby prevent denaturation and destruction of the elastin. (I. Shinji, JP 10-036281, 1998).

To prevent or treat the deterioration of skin quality, consumers have increasingly sought new and/or improved cosmetic/dermopharmaceutical compositions and methods for skin care. These products or methods are designed to prevent, delay or reverse the visible signs of the aging process. And also these products or methods are further designed to improve the appearance and condition of the skin irritated by exposure to potential irritants including chemicals, wind, or sunlight and others. (M. K. Beatrice, U.S. Pat. No. 6,955,816, 2005).

There are several elastase inhibitors already used in the market place as singly or as mixture of compounds including ursolic acid. However, these products are having simultaneous disadvantages associated with each of them. For example ursolic acid is a elastase inhibitor and has not shown any remarkable free radical scavenging property. It is also well known to use extracts from plants and their ingredients as variety of actives to serve such benefit attributes in wide variety of end uses and applications such as medicinal and cosmetic purposes.

The mango is a fruit of the tropical plant Mangifera indica Linn., which belongs to the family Anacardaceae. The plant is widely distributed in Asia, in the Americas and in tropical Africa. Besides the use of the fruit, whether raw or ripe, the utility of different parts of the plant, including the twigs and leaves, bark, resinous gum from the bark, dried flowers, seed kernel, seed fat and sap is described in the botanical and traditional literature of the geographical regions of origin of this plant. The ethnomedical and nutraceutical uses of the diverse forms in which these plant parts can be used are also described in the traditional literature of diverse populations. Some of these properties are listed as anti-asthmatic, antiseptic, antiviral, cardiotonic, emetic, expectorant, hypotensive, and laxative.

The chemicals isolated from the plant fall generally into the classes like aromatic compounds (different classes), amino-acids, carotenoids, carbohydrates, essential oils, enzymes, proteins, fatty acids, flavonoids, glycosides, isoprenoids, carboxylic acids, polyphenolics (different families), polysaccharides, sterols, steroids, vitamins. In particular, the said chemicals include 2-octene, alanine, alpha-phellandrene, alpha-pinene, ambolic-acid, ambonic-acid, arginine, ascorbic-acid, beta-carotene beta-pinene, carotenoids, furfurol, gaba, gallic-acid, gallotannic-acid, geraniol, histidine, isoleucine, isomangiferolic-acid, kaempferol, limonene, linoleic-acid, mangiferic-acid, mangiferine, mangiferol, mangiferolic-acid, myristic-acid, neo-beta-carotene-b, neo-beta-carotene-u, neoxanthophyll, nerol, neryl-acetate, oleic-acid, oxalic-acid, p-coumaric-acid, palmitic-acid, palmitoleic-acid, pantothenic-acid, peroxidase, phenylalanine, phytin, proline, quercetin and xanthophyll, besides others.

Mango skin is generally discarded by humans while eating the fruit, and sometimes provided to animals as feed. It is also reported that compounds like quercetin, mangiferin and norathyriol present in mango skin help fight some metabolic diseases such as diabetes and some forms of cancer in view of their modulation of protein proteosome alpha receptors (www.abc.net.au/science/news). The seed kernel is generally put to veterinary usage. Also, a fat, high in stearic acid content, is known to be isolated from the seed kernel, and butter from the kernel isolate is commercially available to be used to soothe and nourish the skin.

The mango kernel is used as feed for cattle and poultry, as anthelmintic and also as astringent in bleeding files (The Wealth of India, CSIR, New Delhi, 1995 vol. pp 282). Fumes from the burning leaves are inhaled for relief from hiccups and affections from throat. The bark of the tree is used for treating uterine haemorrhage and seed are used in asthma treatment (The useful plants of India, CSIR, New Delhi, 1992, 354.). The fruit is considered very useful in cases of haemorrhage from the uterus, lungs or intestines (Indian medicinal plants, vol. 1, 652).

The mango peel was reported to be good source of dietary fiber containing large amounts of total extractable polyphenolics, which showed high antioxidant property by in-vitro methods (S. Andreas et. al., 3. Agric Food Chem., 2003, 51, 5006-5011). The composition obtained from stem bark of Mangifera indica contains polyphenols, terpenoids, steroids, fatty acids and microelements, which have anti-oxidation, anti-inflammatory, analgesic and anti-spasmodic properties thereby conferring to high value dietary supplements for improve the quality of life of patients suffering from degenerative diseasese, anti-aging treatments (G. P. Oscar et al., CA 235813 A1, 2000). The mangiferin was obtained from the leaf extract, which had shown cardiotonic and diurectic activity (C. Mentzer and A. R. Ratsimamanga, GB 1099764, 1968). Mangiferin has also been indicated as possessing anti-ultraviolet, anti-collagenase and an anti-elastase activity in addition to its anti-free radical and anti-tyrosinase activity. (F. Rouillard et. Al., U.S. Pat. No. 5,824,320, 1998).

No other molecule from Mangifera indica, other than mangiferin or its analogs, has been reported for skin care activity particularly for anti-aging property.

It has now been surprisingly found by way of the present invention that selectively extracts of mango (the fruit of Mangifera indica) skin and/or kernel and/or their active ingredients provide help in combating ageing and for controlling/preventing oxidative damage. Advantageously, the above selective extracts from renewable sources of the invention in cosmetic and/or dermopharmaceutical compositions for skin care are found to provide anti-elastase property. Further such extracts/active ingredients do not contain mangiferin or its analogs as described by F. Rouillard et. al. (U.S. Pat. No. 5,824,320, 1998).

### OBJECTS OF THE INVENTION

It is thus the basic object of the present invention to provide for a skin care agent which would be safe and compatible as a skin care active and favour providing for protection against aging phenomena for skin.

Yet another object of the present invention is directed to a skin care formulation including cosmetic and/or dermopharmaceutical compositions containing the said skin care agent which has attribute such as elastase inhibition property for catering to the needs of variety of skin care conditions and in an effective amount along with an acceptable vehicle with or without other skin benefit agents.

Yet another object of the present invention is to provide a skin care agent providing anti-aging property and which can be sourced simply and cost-effectively for variety of skin care formulations and the like.

A further object of the present invention is directed to a process for the preparation of selective extracts and/or active ingredients from skin/kernel of the fruit of Mangifera indica Linn., and to manner of extracting such selective actives as a skin benefit agent/active especially favouring anti-aging benefits.

A still further object of the present invention is directed to the preparation/formulation of the skin care composition using effective amounts of said extracts and/or active ingredients isolated from skin/kernel of the fruit of Mangifera indica Linn., such that the product obtained by incorporating the active ingredients/extracts have good skin compatibility.

### SUMMARY OF THE INVENTION

Thus according to the basic aspect of the invention there is provided a skin care agent comprising selectively of a polygallate molecule of Formula 1

(R′)₂₋₅—R″ Formula 1

wherein R′=galloyl, R″=polyhydroxy compounds including sugars, as an antiaging agent and any suitable skin care compatible carrier thereof.

According to a further aspect of the invention there is provided a skin care agent comprising selectively a synergistic combination of (a) polygallate molecule of Formula 1

(R′)₂₋₅—R″ Formula 1

wherein R′=galloyl, R″=polyhydroxy compounds including sugars; and
(b) a gallic acid ester of Formula 2

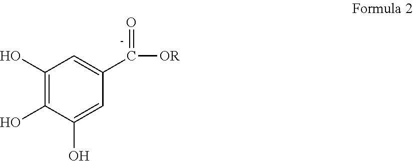

wherein R=alkyl group
in a ratio of (a):(b) of 90:10 to 20:80 and more preferably between 80:20 to 40:60

In accordance with a preferred aspect of the invention, there is provided a skin care agent wherein the said polygallate compound of Formula 1 comprises pentagalloyl glucose of Formula 3 as an active ingredient in effective amounts and the gallic acid ester of Formula 2 comprises methyl gallate of Formula 4 as an active ingredient in effective amounts.

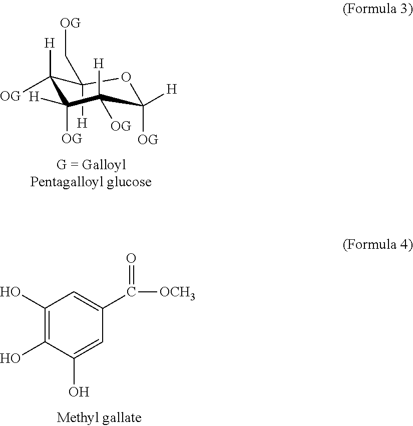

According to another aspect of the invention there is provided a cosmetic/dermopharmaceutical composition for topical use comprising an effective amount of mixture of compounds of Formula 1 and Formula 2

and
a cosmetically/dermopharmaceutically acceptable vehicle with or without other skin benefit agents.

A further aspect of the present invention is a skin care formulation including cosmetic and/or dermopharmaceutical compositions comprising effective amounts of selective extracts and/or active fractions derived from skin and/or kernel of fruit of Mangifera indica possessing anti-elastase character.

In accordance with yet another aspect of the invention there is provided a cosmetic and/or dermopharmaceutical composition for topical use comprising an effective amount of a mixture comprising of compounds of Formula 3 and Formula 4 as an active ingredient sourced from Mangifera indica displaying substantially the following cnaractenstics on min layer chromatography using a pre-coated silica gel plate, the isolated substance of Formula 3 provides a spot with R_{f} value of 0.51 in EtOAc: MeOH 8:2; the UV(MeOH) spectrum showed an absorbance value at 281 nm; IR spectrum values are at 3410 (br), 1716 and 1707 cm⁻¹ and other characteristic signals; Proton NMR values in DMSO-d₆ are: δ 6.96(2H, s), 6.90 (2H, s), 6.84 (2H,$), 6.80 (2H, s), 6.75 (2H, s), 6.37 (1H, d), 5.94 (1H, t), 5.42 (2H, over lapped triplet), 4.59 (1H, d), 4.29 (2H, s) and the isolated substance, Formula 4 provides a spot with an R_{f} value of 0.44 in CHCl₃: EtOAC 8:2; the UV(MeOH) spectrum showed an absorbance value at 281 nm; IR spectrum values are at 3450 (br), 1743 cm⁻¹ and other characteristic signals; Proton NMR values in CD₃OD are: 63.6 (3H, s), 7.02 (2H, s).

and
a cosmetically acceptable vehicle with or without other skin benefit agents.

According to a preferred aspect of the cosmetic/dermopharmaceutical composition for topical use comprises:
a. about 0.0001 wt % to about 20 wt. % of the said skin care agent, preferably 0.001 wt % to 10 wt % of the said skin care agent and more preferably and 0.01 wt % to 5 wt % of the said skin care agent as an active ingredient and
b. a cosmetically acceptable vehicle with or without other skin-benefit agents.

According to yet further aspect of the invention, the said cosmetic/dermopharmaceutical composition for topical use can be a leave on or a wash-off product adapted for topical delivery in the form of creams, ointments, emulsions, gels, lotions, oils, sticks, sprays, soaps, packs, wraps, woven or nonwoven wipes, films or patches as a vehicle for topical application of skin care composition.

According to yet further aspect of the invention there is provided a cosmetic/dermopharmaceutical composition for topical use wherein said skincare composition consisting of a compound of Formula 1 or a mixture of compounds of Formula 1 and Formula 2 or preferably pentagalloyl glucose of Formula 3 or a mixture of pentagalloyl glucose of Formula 3 and methyl gallate of Formula 4, as concentrate of an appropriate natural isolate comprising preferably compound of Formula 1 or a mixture of compounds of Formula 1 and Formula 2 or more specifically pentagalloyl glucose of Formula 3 or a mixture of pentagalloyl glucose of Formula 3 and methyl gallate of Formula 4, an extract/concentrate of the natural material or even natural material itself.

According to a further aspect of the invention, the said cosmetic/dermopharmaceutical composition for topical use includes extract/concentrate from skin/seed kernel of Mangifera indica Linn., preferably methanol concentrate or more preferably ethyl acetate concentrate containing mixture of polygalloyl glucose of Formula 3 and methyl gallate of Formula 4.

In accordance with yet another aspect of the invention there is provided a process for the extraction of Mangifera indica comprising:
a) subjecting the air-dried fruit skin/kernel of Mangifera indica Linn., in powder form, to extraction with an organic solvent, preferably ethyl acetate/methanol, to provide an extract,
b) concentrating the extract in vacuo to provide a concentrate,
c) subjecting residue to column chromatography, preferably a silica gel column, using as eluents solvents such as ethyl acetate:chloroform (3:1), ethyl acetate, ethyl acetate:methanol (3:1) to provide fractions,
d) subjecting the fractions to bioactivity screening in an elastase inhibition activity screening assay to provide active fractions,
e) pooling the active fractions and concentrating the resulting solution to dryness in vacuo to provide another concentrate,
f) subjecting the concentrate to further chromatography and obtain more enriched concentrate
g) subjecting the enriched concentrate to a process of crystallization to provide the compounds of the invention.

In another aspect the present invention relates to a process of preparing the selective extract and/or active ingredient derived from skin and for kernel extracts of the fruit of the plant Mangifera indica to be used in the composition of the invention.

Yet another aspect of the present invention relates to the process for preparing the selective extract and/or active ingredients comprising their extraction from the skin and/or kernel of the fruit of Mangifera indica fruit using appropriate solvent and sequence of operations. The isolation process may result in mixture of compounds having a different and defined proportion or in an enriched extract high in specific compound/s or in a pure compound.

A further aspect of the present invention relates to process for making and using the compositions which comprise an effective amount of a selective extract and/or active ingredient isolated from the skin and/or kernel of the fruit of Mangifera indica together with cosmetically/dermopharmaceutically acceptable vehicles. Methods are described for making and using the cosmetic compositions particularly suited for application to achieve anti-aging effect.

The above discussed cosmetic/dermopharmaceutical composition for topical use can be a leave-on or a leave-off products wherein the delivery system comprises creams, ointments, emulsions, gels, lotions, oils, sticks, sprays, soaps, packs, wraps, woven or non-woven wipes, films or patches as vehicle for topical application of the said skin care composition.

The term “skin” as used herein includes epithelial tissue (epiderrrial layer) naturally exposed to the environment and can be the face, neck, chest, back, arms, axillae, hands, legs, abdomen, buttocks, groin, feet, scalp, etc.

The term “cosmetic and/or dermopharmaceutical composition” is intended to describe compositions for topical application by way of applying and spreading on to the surface of human skin, including leave-on and wash-off products.

The term “cosmetically/dermopharmaceutically acceptable” as used herein means that the compositions/compounds so described are suitable for prolonged contact with human skin without undue toxicity, allergic responses, incompatibility, instability etc.

The term “effective amount” as used herein means that an amount of the compositions/compounds so described is sufficient to induce a positive/desirable benefit without causing any unacceptable/serious side effect.

The term “compatibility” as used herein means that the compositions/compounds so described are suitable for prolonged contact with nil or acceptable adverse skin reactions such as stinging, erythema, itching, burning sensation etc.

Except in the examples, or where otherwise explicitly indicated, all numbers in this description indicating amounts of materials or conditions of reaction, physical properties of materials and/or use are to be understood as modified by the word “about”. All amounts are by weight of the compositions, unless otherwise specified.

For the avoidance of doubt, the term “comprising” means including, made up of, composed of, consisting and/or consisting essentially. Furthermore, in the ordinary meaning of “comprising,” the term is defined as not being exhaustive of the steps, compositions, ingredients, or features to which it refers.

### DETAILED DESCRIPTION OF THE INVENTION

The present invention provides a single compound or mixture of compounds as extract or pure compounds acting as anti-aging and free radical scavenging agent in a cosmetic and dermopharmaceutical composition for topical use, to process for producing the said mixture or pure compounds by isolation from skin and/or kernel of the fruit of Mangifera indica Linn., and to processes for producing and using the said compositions to achieve anti-aging effect.

According to a preferred aspect of the invention the skin care formulation including cosmetic and/or dermopharmaceutical compositions comprises:
a. about 0.0001% to about 20% weight percent of a mango skin and kernel extract and/or active ingredient; and
b. a cosmetically acceptable vehicle.

The amount of the mango skin and kernel extract/concentrate and/or active ingredient is preferably in the range of about 0.0001% to about 20%, more preferably about 0.001% to 10%, most preferably 0.01% to about 5% of the total amount of the cosmetic composition.

Importantly, the selective extract/concentrate of the skin and kernel extracts of the fruit of the plant Mangifera indica according to the invention generally comprise as active ingredients classes of chemicals preferably selected from aromatic compounds, amino-acids, carotenoids, essential oils, enzymes, fatty acids, flavonoids, glycosides, isoprenoids, carboxylic acids, polyphenolics (different families), mono or polysaccharides, proteins, sterols, steroids, vitamins.

According to a preferred aspect of the invention there is provided a skin care formulation including cosmetic and/or dermopharmaceutical compositions which comprise skin and/or kernel extract/concentrates of the fruit of the plant Mangifera indica which comprise the aforementioned selective group of chemical classes and group of particular chemicals.

In accordance with yet another aspect of the invention there is provided a method of extraction of selective extracts from the skin of the raw and/or ripe mango and of the seed kernel of the mango fruit of the plant. For the purposes of extraction of such selective actives suitable conventional extraction methods, such as maceration, remaceration, agitation maceration, digestion, fluidized-bed extraction, ultrasound extraction, countercurrent extraction, cryogenic extraction, percolation, repercolation, evacolation, diacolation and solid-liquid extraction under continuous reflux which is carried out in a Soxhlet extractor can be carried out, each of which is known to the person skilled in the art and any of which can be used in principle, reference may be made by way of example to Hagers Handbuch der Pharmazeutischen Praxis (5.sup.th edition, Vol. 2, pp. 1026 1030, Springer Verlag, Berlin-Heidelberg-New York 1991).

Importantly, the selective starting materials for the extraction are fresh or dried skin and kernel of the mango fruit of the plants, which can be mechanically comminuted prior to extraction. Preferably, coarsely ground air-dried skin of the fruit of Mangifera indica or coarsely ground air-dried seed kernels of Mangifera indica or air-dried seed kernels of Mangifera indica cut into small pieces were subjected to extraction with one or more organic solvents like lower alcohols, preferably methanol, or esters, preferably, ethyl acetate or halogenated hydrocarbons, preferably chloroform, to provide an extract.

In this connection, all comminution methods known to the person skilled in the art are suitable, mention being made by way of example to comminution using a device containing blades.

In accordance with a preferred aspect of the invention, after the extraction, the resulting crude extracts is optionally subjected to further customary steps, such as, for example, concentration, fractionation by partitioning between solvents, purification, including different types of chromatography, concentration and/or decoloration. If desired, the extracts prepared in this way can, for example, be subjected to selective removal of individual undesired ingredients. The extraction can be carried out to any desired degree of extraction, but is usually carried out exhaustively.

Importantly in accordance with yet further aspect of the invention the present invention encompasses the finding that the extraction conditions and also the yields of the end extracts are selectively chosen depending on the desired field of use, by way of evaluation of efficacy through deployment of appropriate precise-screening technique, including anti-elastase activity, and other related screens applicable to the benefit attributes in accordance with the present invention and skin care formulations incorporating the same.

In accordance with yet another preferred aspect of the invention, the selective extract was concentrated in vacuo to provide a concentrate, the concentrate was subjected to one or more separation techniques to thereby identify the actives for the desired efficacy in accordance with the skin care formulation of the invention.

Preferably, according to another aspect of the invention a typical active substance of the invention, whether obtained from the skin of the fruit or from the seed kernel comprised a combination of compounds.

Mangifera indica: The Selective Natural Renewable Source for the Extract/concentrates of the Invention.

The extracts to be used according to the invention are obtained from the skin and/or kernel of the mango fruit of a plant of the Anacardaiceae family, specifically Mangifera indica.

### Extract/concentrates

The skin and kernel extract/concentrates of the mango fruit of the plant Mangifera indica according to the present invention generally comprise, as active ingredients, classes of chemicals like aromatic compounds (different classes), amino-acids, carotenoids, carbohydrates, essential oils, enzymes, fatty acids, flavonoids, glycosides, isoprenoids, carboxylic acids, polyphenolics (different families), polysaccharides, proteins, sterols, steroids, vitamins. The final product has a composition which varies depending on the staring material and on the method of extraction chosen.

### The Solvent Extraction Process:

The extract/concentrates used according to the invention as described above for the purpose of producing desired multifunctional skin care formulation, are prepared by conventional methods of extraction of the skin of the raw and/or ripe mango and of the seed kernel of the mango fruit of the plant. Such suitable conventional extraction methods, may include maceration, remaceration, agitation maceration, digestion, fluidized-bed extraction, ultrasound extraction, countercurrent extraction, cryogenic extraction, percolation, repercolation, evacolation, diacolation and solid-liquid extraction under continuous reflux which is carried out in a Soxhlet extractor, each of which is known to the person skilled in the art and any of which can be used in principle, reference may be made by way of example to Hagers Handbuch der Pharmazeutischen Praxis (5.sup.th edition, Vol. 2, pp. 1026 1030, Springer Verlag, Berlin-Heidelberg-New York 1991).

Starting material are fresh or dried skin and kernel of the mango fruit of the plants Mangifera indica, which are used for extraction of active ingredients and can be mechanically comminuted prior to extraction. In this connection, all comminution methods known to the person skilled in the art are suitable, mention being made by way of example to comminution using a device containing blades.

### Solvents:

Solvents used for carrying out the extractions are preferably organic solvents, water or mixtures of organic solvents and water, in particular low molecular weight alcohols, esters, ethers, ketones or halogen-containing hydrocarbons with greater or lesser water contents (distilled or un-distilled), preferably aqueous, alcoholic solutions with greater or lesser water contents. However, preference is particularly given to the extraction with water, methanol, ethanol, propanol, butanol and isomers thereof, acetone, methyl ethyl ketone, propylene glycols, polyethylene glycols, ethyl acetate, chloroform, dichloromethane, trichloromethane, and mixtures thereof.

The extraction usually takes place at 20° C. to 140° C., preferably at 30° C. to 120° C., in particular at the boiling temperature of the solvents or solvent mixtures. In one embodiment, the extraction is carried out under an inert gas atmosphere to avoid oxidation of the ingredients of the extract. The extraction times are adjusted by any person skilled in the art depending on the variable parameters for the extraction process such as starting material, the extraction method, the extraction temperature, the ratio of solvent to raw material, etc. After the extraction, the resulting crude extracts can optionally be subjected to further customary steps, such as, for example, concentration, fractionation by partitioning between solvents, purification, including different types of chromatography, concentration and/or decoloration. If desired, the extracts prepared in this way can, for example, be subjected to selective removal of individual undesired ingredients. The extraction can be carried out to any desired degree of extraction, but is usually carried out exhaustively.

Air-dried skin and seed kernel of the fruit of Mangifera indica, preferably, coarsely ground, or air-dried seed kernels of Mangifera indica cut into small pieces, are subjected to extraction with one or more organic solvents like lower alcohols, preferably methanol, or esters, preferably, ethyl acetate or halogenated hydrocarbons, preferably chloroform, to provide an extract.

The present invention encompasses the finding that the extraction conditions and also the yields of the end extracts can be chosen depending on the desired field of use, through deployment of appropriate precise screening techniques, including anti-elastase activity and other related screens.

The Active Ingredient/s from Mangifera indica Linn.,

The methanolic extract of the skin/seed kernel was concentrated in vacuo to provide a concentrate. The concentrate was subjected to one or more separation techniques usually used by those skilled in the art, in particular partitioning the concentrate between different organic solvents and using chromatographic techniques such as column chromatography, always monitoring the resulting fractions in bioactivity screening assays to trace the progression of the extracts displaying activity in order to provide an active substance of the invention. A typical active fraction of the invention, whether obtained from the skin of the fruit or from the seed kernel comprised a mixture of compounds when analyzed by high pressure liquid chromatography(HPLC)-reverse phase column, RP-18 with distinct retention times (16.72, 41.63T) in binary mobile phase system, 2% AcOH in water and 0.5% AcOH in 50:50 acetonitirile: H₂O). The UV spectrum of a methanolic solution has shown characteristic absorption maxima at 279 nm.

As stated herein before, the isolated substance from Mangifera indica Linn., for use in the composition of the invention is found to relate to mixture of at least one polygallate derivative of pentagalloyl glucose and methyl gallate. The compound of Formula 3 provides a spot with R_{f} value of 0.51 in EtOAc: MeOH 8:2; the UV(MeOH) spectrum showed an absorbance value at 281 nm; IR spectrum values are at 3410 (br), 1716 and 1707 cm⁻¹ and other characteristic signals; Proton NMR values in DMSO-d₆ are: δ 6.96(2H, s), 6.90 (2H, s), 6.84 (2H, s), 6.80 (2H, s), 6.75 (2H, s), 6.37 (1H, d), 5.94 (1H, t), 5.42 (2H, overlapped t), 4.59 (1H, d), 4.29 (2H, s) and methyl gallate derivative of Formula 4 of the invention displays the following characteristics. On thin layer chromatography using a pre-coated silica gel plate (Merck 1.05554.0001), the isolated substance provides a spot with an R_{f} value of 0.44 in CHCl₃: EtOAC 8:2; the UV(MeOH) spectrum showed an absorbance value at 281 nm; IR spectrum values are at 3450 (br), 1743 cm⁻¹ and other characteristic signals; Proton NMR values in CD₃OD are: 63.6 (3H, s) 7.02 (2H,$).

The thus observed R_{f} values and spectroscopic features are observed to support that the isolated substances conform to at least one gallate ester of the Formula 2 and a polygallate compound of Formula 1.

The synergistic mixture of pentagalloyl glucose of Formula 3 and methyl gallate of Formula 4 is used in cosmetic and/or dermopharmaceutical compositions for topical use either as a pure compound or as a part of a mixture of compounds in an effective amount or as an extract from a suitable source such as fruit skin/or seed kernel of the plant Mangifera indica Linn., in an effective amount along with a cosmetically/dermopharmaceutically acceptable vehicle.

### Care Agents

For the purposes of the Invention, care agents are understood as meaning care agents for skin. These care agents include, inter alia, cleansing and restorative action for skin.

Applications are topical in the form of creams, lotions, solutions, gels, sprays or any other form that can be made and used over a surface.

Moreover, the preparations according to the invention exhibit an excellent skincare action coupled with simultaneously high skin compatibility. The preparations have a large number of cosmetic and/or dermopharmaceutical effects. The invention therefore further provides for the incorporation of extracts from the skin and kernel of the mango fruit of the plant, Mangifera indica, against skin aging, in particular against wrinkling and line formation.

The said extract/concentrates according to the invention, from the skin and kernel of the mango fruit of the plant, Mangifera indica, also act as protease-inhibiting agent, in particular as elastase-inhibiting agent. The elastase which occurs predominantly belongs to the group of serine proteases. These proteases (collagenase and the various elastases) catalyze the fragmentation and destruction of the dermal macromolecules, such as proteoglycan, collagen and elastin, and thereby lead to aging of the skin and to the effects of natural skin aging following UV radiation.

The present invention is also directed to said selective extract/concentrate from the skin and kernel of the mango fruit of the plant Mangifera indica acting as an antioxidant or free-radical scavenger.

For the purposes of the invention, antioxidants are understood as meaning oxidation inhibitors which can be isolated from the skin and kernel of the mango fruit. Antioxidants are able to inhibit or prevent the undesired changes in the substances to be protected caused by oxygen effects and other oxidative processes. The effect of the antioxidants consists in most cases in them acting as free-radical scavengers for the free radicals which arise during autoxidation.

As well as the use of extract/concentrates of the skin and kernel of the mango fruit of the plant Mangifera indica as antioxidants, further, already known antioxidants can also be used. A possible use of the antioxidants for example in cosmetic and/or dermopharmaceutical preparations is the use as secondary light protection agents, since antioxidants are able to interrupt the photochemical reaction chain which is triggered when UV radiation penetrates the skin. In addition to the use of the plant extract according to the invention, additional typical usable secondary light protection agents can also be used. The further amounts of UV light protection factors or antioxidants can be added in amounts of from 0.01 to 25% by weight, preferably 0.03 to 10% by weight and in particular 0.1 to 5% by weight, based on the total amount in the preparations.

The use of the extracts according to the invention as protective and restorative care agents is obtainable for all preparations which are used for prevention against damage or in the case of damage to the skin, thus in skincare. Another use in this field is the application in cases of sensitive skin damaged by allergy or other causes. The damage to the skin can have a very wide variety of causes.

The composition according to the invention can be used for the production of cosmetic and/or dermopharmaceutical preparations, such as, for example, lotions, foam baths, shower baths, creams, gels, sprays, lotions, alcoholic and aqueous/alcoholic solutions, emulsions, wax/fat compositions, stick preparations or ointments. These preparations can also comprise, as further auxiliaries and additives, surfactants, oily bodies, emulsifiers, pearlescent waxes, bodying agents, thickeners, superfatting agents, stabilizers, polymers, silicone compounds, fats, waxes, lecithins, phospholipids, biogenic active ingredients, deodorants, antiperspirants, film formers, swelling agents, hydrotropes, solubilizers, preservatives, perfume oils, dyes and the like.

The skin care composition of the invention can also incorporate conventional cosmetic and skin care ingredients such as discussed hereunder:

The various components usually used for cosmetic composition, i.e., an aqueous component, an oily component, a powder component, alcohols, ester, a surfactant, a moisturizer, a whitening agent, an antioxidant, an ultraviolet ray absorbent, a thickener, a coloring material, perfume, an anti-oxidant, pH regulator, a chelating agent, and antiseptics, can be blended with these cosmetics.

The ingredients essentially employed in such a composition are surfactants, oil bodying agents, emulsifiers, fats and waxes, stabilizers, polymers, silicone compounds, deodorants and antimicrobial agents, film formers, swelling agents preservatives, Thickeners/herbal extracts/concentrates/active ingredients, pH adjusting agents, perfumes oils, and water to qs.

The composition may contain usually employed vehide such as may be aqueous, anhydrous or an emulsion. Preferably, the compositions are aqueous or an emulsion, especially water-in-oil or oil-in-water emulsion, preferentially oil in water emulsion. Water when present will be in amounts which may range from 5 to 99%, preferably from 20 to 85%, optimally between 40 and 80% by weight.

### Surfactants

Surface-active substances which may be present are anionic, nonionic, cationic and/or amphoteric surfactants, the content of which in the compositions is usually about 1 to 70% by weight, preferably 5 to 50% by weight and in particular 10 to 30% by weight.

### Oily Bodies

Suitable oily bodies can also be incorporated which include for example, Guerbet alcohols based on fatty alcohols having 6 to 18, preferably 8 to 10, carbon atoms, esters of linear C₆-C₂₂-fatty acids with linear or branched C₆-C₂₂-fatty alcohols or esters of branched C₆-C₁₃-carboxylic acids with linear or branched C₆-C₂₂-fatty alcohols. Such oily bodies can be used in amounts of 0.1 to 50.0% by wt.

### Emulsifiers

Suitable emulsifiers are, for example, nonionic surfactants.

The addition products of ethylene oxide and/or of propylene oxide onto fatty alcohols, fatty acids, alkylphenols or onto castor oil are also known, commercially available products which can be included.

Alkyl and/or alkenyl oligoglycosides, their preparation and their use are known from the prior art.

Furthermore, zwitterionic surfactants can be used as emulsifiers. The term “zwitterionic surfactants” refers to those surface-active compounds which carry at least one quaternary ammonium group and at least one carboxylate and one sulfonate group in the molecule. Likewise suitable emulsifiers are ampholytic surfactants. The term “ampholytic surfactants” means those surface-active compounds which, apart from a C.sub.8/18-alkyl or -acyl group in the molecule, contain at least one free amino group and at least one —COON or —SO₃H group and are capable of forming internal salts. Finally, cationic surfactants are also suitable emulsifiers, those of the ester quat type, preferably methyl quaternized difatty acid triethanolamine ester salts, being particularly preferred.

Such emulsifiers can be used in amounts of 0.1 to 50.0% by wt.

### Fats and Waxes

Typical examples of fats are glycerides, i.e. solid or liquid vegetable or animal products which consist essentially of mixed glycerol esters of higher fatty acids, suitable waxes are inter alia natural waxes. In addition to the fats, suitable additives are also fat-like substances, such as lecithins and phospholipids. The term lecithins is understood by the person skilled in the art as meaning those glycerophospholipids which form from fatty acids, glycerol, phosphoric acid and choline by esterification. Lecithins are thus frequently also known as phosphatidylcholines (PC). Examples of natural lecithins which may be mentioned are the cephalins, which are also referred to as phosphatidic acids and represent derivatives of 1,2-diacyl-sn-glycerol-3-phosphoric acids. By contrast, phospholipids are usually understood as meaning mono- and, preferably, diesters of phosphoric acid with glycerol (glycerophosphates), which are generally considered to be fats. In addition, sphingosines and sphingolipids are also suitable. Such fats and waxes can be used in amounts of 0.1 to 10% by wt.

Moreover, known. Pearlescent Waxes, Bodying Agents and Thickeners as well as superfatting agents can also be used.

Superfatting agents which can be used are substances such as, for example, lanolin and lecithin, and polyethoxylated or acylated lanolin and lecithin derivatives, polyol fatty acid esters, monoglycerides and fatty acid alkanolamides, the latter also serving as foam stabilizers. Such super fatting agents can be used in amounts of 0.1 to 15% by wt.

### Stabilizers

Stabilizers which can be used are metal salts of fatty acids, such as, for example, magnesium, aluminum and/or zinc stearate or ricinoleate. Such stabilizers can be used in amounts of 0.1 to 10% by wt.

### Polymers

Suitable polymers include anionic, zwitterionic, amphoteric, nonionic and cationic polymers. Suitable polymers are, for example, suitable polymers and thickeners are listed in Cosm. Toil. 108, 95 (1993). Such polymers can be used in amounts of 0.001 to 5% by wt.

### Silicone Compounds

Suitable silicone compounds which are volatile or non-volatile, for example, dimethylpolysiloxanes, methylphenylpolysiloxanes, linear and cyclic silicones, and amino-, fattyacid-, alcohol-, polyether-, epoxy-, fluorine-, glycoside- and/or alkyl-modified silicone compounds, which can either be liquid or in resin form at room temperature. Also suitable are simethicones, which are mixtures of dimethicones having an average chain length of from 200 to 300 dimethylsiloxane units and hydrogenated silicates. Such silicone compounds can be used in amounts of 0.1 to 10% by wt.

### Biogenic Active Ingredients

Biogenic active ingredients can also be included meaning those which do not arise from the plant Mangifera indica, such as, for example, tocopherol acetate, tocopherol palmitate, ascorbic acid, (deoxy) ribonucleic acid and fragmentation products thereof, retinol, bisabolol, allantoin, phytantriol, panthenol, AHA acids, amino acids, ceramides, pseudoceramides, essential oils, further plant extracts and additional vitamin complexes. Such biogenic active ingredients can be used in amounts of 0.001 to 5.0% by wt.

### Deodorants and Antimicrobial Agents

Cosmetic deodorants are also possible ingredients which can be added to counteract, mask or remove body odors. Body odors arise as a result of the effect of skin bacteria on apocrine perspiration, with the formation of degradation products which have an unpleasant odor. Accordingly, deodorants comprise active ingredients which act as antimicrobial agents, enzyme inhibitors, odor absorbers or odor masking agents.

Antiperspirants can also be added in the cosmetic preparation to reduce the formation of perspiration by influencing the activity of the eccrine sweat glands, thus counteracting underarm wetness and body odor.

Suitable astringent antiperspirant active ingredients are primarily salts of aluminum, zirconium or of zinc.

Customary water-soluble additives are, for example, preservatives, water-soluble fragrances, pH regulators, e.g. buffer mixtures, water-soluble thickeners, e.g. water-soluble natural or synthetic polymers, such as, for example, xanthane gum, hydroxyethylcellulose, polyvinylpyrrolidone or high molecular weight polyethylene oxides.

Such deodorant and antimicrobial agents can be used in amounts of 0.001 to 5.0% by wt.

### Film Formers

Conventionally the film formers are selected from, for example, chitosan, microcrystalline chitosan, quaternized chitosan, polyvinylpyrrolidone, vinylpyrrolidone—vinyl acetate copolymers, polymers of the acrylic acid series, quaternary cellulose derivatives, collagen, hyaluronic acid and salts thereof, and similar compounds. Suitable film formers can be used in amounts of 0.001 to 5.0% by wt.

### Thickeners

Thickeners/rheology modifiers may also be utilized as part of the cosmetically acceptable carrier of compositions according to the present invention. Amounts of the thickener may range from 0.0001 to 10%, usually from 0.001 to 5%, by weight

### Swelling Agents

The swelling agents for aqueous phases may be montmorillonites, clay mineral substances, Pemulen, and alkyl-modified Carbopol grades (Goodrich). Such additives can be incorporated in amounts of 0.001 to 2.0% by wt.

### Hydrotropes

To improve the flow behavior, hydrotropes can be added, such as, for example, ethanol, isopropyl alcohol, or polyols, can also be used. Such additives can be incorporated in amounts of 0.1 to 15% by wt.

### Preservatives

Suitable preservatives are, for example, phenoxyethanol, formaldehyde solution, parabenes, pentanediol or sorbic acid, and the other classes of substance listed in Annex 6, Part A and B of the Cosmetics Directive. Such preservatives can be incorporatea in amounts of 0.001 to 5% by wt.

Other adjunct minor components may also be incorporated into the cosmetic compositions. These ingredients may include coloring agents, opacifiers, and perfumes. Amounts of these other adjunct minor components may range anywhere from 0.001% up to 20% by weight of the composition.

It is thus possible by way of the present invention to provide for skin care formulation including cosmetic and/or dermopharmaceutical compositions for skin care involving selective extract of skin and kernel of the mango fruit of the plant Mangifera indica which would on one hand provide care and vitalizing effect such as to obtain protection against aging phenomena for skin and also antioxidant/sunscreen effects, on the other hand would simultaneously have a positive influence on or at least do not impair the technical properties of the cosmetic product such as the storage stability, photostability and ability to be formulated. The invention is thus directed to meet consumer-demands in skin care including separate expectation of the consumers relating to the aspect of good skin compatibility and particularly the use of natural products in the preparation/formulation. Advantageously, the cosmetic and/or dermopharmaceutical compositions for skin care of the invention would avoid the need for combining too many varied and complex active ingredients and avoid the complexity of the formulation yet favour several diverse attributes such as anti-elastase and antioxidant properties in a balanced form for catering to the varied needs of a skin care formulation.

The composition according to the invention involving the above skincare composition is intended primarily as a product for topical application to skin for the purpose of c anti-aging activity. In use, a small quantity of the composition, for example about 0.1 to about 5 ml/g, can be applied to exposed areas of the skin, from a suitable container or applicator and, if necessary, it is then spread over and/or rubbed into the skin using the hand or fingers or a suitable device for desired skin benefit activity.

The details of the invention, its objects and advantages are explained hereunder in greater detail in relation to non-limiting exemplary illustrations as per the following examples:

### EXAMPLES

### Example 1

### Extraction, Purification and Isolation of Active Compounds from Skin of M. indica

The Air-dried skin of Mangifera indcia (200 g) was coarsely powdered, extracted with methanol through Soxhlet apparatus for about 8 hrs. The solvent was left overnight in the apparatus. Next day it was concentrated under reduced pressure to get 46 g of concentrate. The concentrate was dispersed in water:methanol (1:1) in 200 ml. The aq. methanolic solution was fractionated successively with chloroform (300 ml×3), ethyl acetate (200 ml×3) and n-butanol which was saturated with water (300 ml×3) to get corresponding fractions 7 g, 4 g, and 24 g respectively. The ethyl acetate fraction was found to be most effective for anti-aging activity (IC₅₀=4.2 μg/ml) comprising a mixture of two compounds of Formula 3 and Formula 4 in different ratios.

The ethyl acetate fraction (4 g) was dissolved in 6 ml of methanol and adsorbed on 5 g of silica gel (100-200 mesh). 70 g of silica gel was packed in the glass column by using ethyl acetate:chloroform (1:1) as solvent and adsorbed silica gel was loaded on top the column. The column was eluted with solvent mixture of chloroform: ethyl acetate (1:1, 1:3) and finally eluted with ethyl acetate and collected 58 fractions (each about 15 ml). Combined homogeneous fractions and divided into four fractions. Two fractions, Fr.3 and Fr.4 demonstrated anti-aging activity and Fr. 3 was found to be more potent (IC₅₀=1.9 μg/ml) than other fraction comprising a mixture of two compounds of Formula 3 and Formula 4 in different ratios which is not again similar to the abovesaid ratio of compounds of Formula 3 and Formula 4 in the ethyl acetate fraction with (IC₅₀=4.2 μg/ml).

### Example 2

### Extraction of Active Compounds from Kernel of M. indica

The Air-dried kernels of Mangifera indcia Linn., (400 g) were extracted with methanol through soxhlet apparatus for about 16 hrs. The methanolic extract was concentrated under reduced pressure to get 45 g of concentrate. The concentrate (44 g) was dispersed in 200 ml of water:methanol (4:1). The aqueous methanolic solution was fractionated with chloroform, ethyl acetate and n-butanol which saturated with water to get corresponding fractions, 20.0, 23.0 and 3.0 g respectively. The ethyl acetate fraction was found to be more potent (IC₅₀=2.97 ug/ml) than chloroform and butanolic fractions. This fraction was found to be mixture of two compounds of Formula 3 and Formula 4.

### Example 3

### Biologial Assays

This example demonstrates the method of determining the elastase inhibition activity of the specially preferred concentrates/compounds/formulations of the invention by elastase inhibition method to estimate the reduction of elastase.

### a) Elastase Inhibition Assay

300 μl (0.6 mg) of succinyl-L-alanyl-L-alanyl-L-alanyl-p-nitroanilide (the enzyme substrate), 1200 μl of buffer and varying amounts of the elastase inhibitor under testing are incubated at 37° C. for 20 minutes. The hydrolysis is measured by the spectrophotometric measurement of the release of p-nitroaniline at a wavelength of 410 nm. In this method, the concentrates of skin, kernel, compounds and formulations demonstrate the results and are mentioned in Table.1

TABLE 1

| Concentrate/compound/Formulation | Inhibition |
|---|---|
| Ursolic acid (Sigma cat. no: U-6753) | IC₅₀ = 13.1 μg/ml |
| Mango skin concentrate | 36% at 59.7 μg/ml |
| Mango seed kernel concentrate | IC₅₀ = 2.48 μg/ml |
| Ethyl acetate fraction from mango skin | IC₅₀ = 4.2 μg/ml |
| Ethyl acetate fraction from mango seed kernel | IC₅₀ = 2.97 ug/ml |
| Formula 4 | 11.15% at 43.5 μg/ml |
| Formula 3 | 56.6% at 70 μg/ml |
| Skin cream | 59.25% at 40 μg/ml |
| Skin lotion | 21.3% at 40 μg/ml |

### b) Synergistic Studies of Pentagalloyl Glucose of Formula 3 and Methyl Gallate of Formula 4

The anti-aging activity of the individual molecules along with different combinations were studied and the results mentioned in Table.2

TABLE 2

| Pentagalloyl glucose:Methyl gallate | % Inhibition |
|---|---|
| 100:0 | 49 |
| 80:20 | 64.5 |
| 60:40 | 58.7 |
| 40:60 | 43.9 |
| 20:80 | 31.7 |
| 0:100 | 3.0 |

### Example 4

### Formulation of Skin Cream

TABLE 3

| | | Quantity in |
|---|---|---|
| Phase | Parameter | g/100 g |
| A | Stearic aicd | 14.00 |
| | Cetyl alcohol | 0.50 |
| B | Potassium hydroxide | 0.50 |
| | DM water | 2.00 |
| C | Titanium dioxide | 0.20 |
| | Glycerin | 1.00 |
| | DM water | 66.60 |
| D | Isopropyl stearate | 2.00 |
| | Octyl methoxy cinnamate | 1.50 |
| | Avobenzone | 0.40 |
| | Benzophenone | 0.10 |
| | Propylparabenzoate | 0.10 |
| | DC-200 (350 CST) | 1.00 |
| E | Ethylene diammine tetraactetic acid | 0.10 |
| | Disodium salt | |
| | DM water | 2.0 |
| F | Methyl parabenzoate | 0.25 |
| | Propylene glycol | 1.00 |
| G | DC-25 | 1.00 |
| | Vitamin E acetate | 0.1 |
| H | DM Red 33 (1% soln) | 0.05 |
| | Sunset yellow (1% soln) | 0.10 |
| | Perfume | 0.50 |
| I | Concentrate | 2.00 |
| | Alcohol | 3.00 |

1. Heat Phase A to 75 degrees C. on water bath and melt the mixture
2. Heat Phase B to 75 degrees C. in a separate container.
3. Disperse Phase C material and heat to 75 degree C. on water bath. Add Phase B to C, then add Phase A to Phase ‘BC’ and stir for 5 minutes.
4. Heat Phase D mixture to 75 degree C., add to Phase ABC at high speed and emulsify the mixture for about 15 minutes.
5. Dissolve EDDA-DS from Phase E and add to Phase ABCD.
6. Add Phase F ingredients at 75 degree C. and stir for 5 minutes. Cool the material to 45 degree C. by ordinary cooling system.
7. Add Phase G material at 45 degree C. to Phase ABCDEF.
8. Add Phase H materials to Phase ABCDEFG.
9. Dissolve the active fraction in alcohol and add to Phase ABCDEFGH and stir for 5 minutes.

### Example 4

### Formulation of Skin Lotion

TABLE 4

| | | Quantity in |
|---|---|---|
| Phase | Parameter | g/100 g |
| A | Light liquid paraffin | 6.00 |
| | Octyl methoxy cinnmate | 1.00 |
| | Avobenzone | 0.20 |
| | Benzophenone | 0.10 |
| | Isopropyl myristate | 2.00 |
| | Cetyl alcohol | 1.50 |
| | Stearic acid | 1.0 |
| | Ethylene glycol distearate | 1.00 |
| | Propyl parabenzoate | 1.50 |
| | DC-200 | 0.40 |
| | Butylated hydroxy toluene | 0.10 |
| | Arlacel 60 | 0.10 |
| | Tween 60 | 1.00 |
| B | Glycerine | 1.00 |
| | Titanium dioxide | 0.10 |
| | Di sodium ethylene diamine tetraacetate | 0.05 |
| | Methyl parabenzoate | 0.25 |
| | Deminaralized water | 48.33 |
| C | Carbopol 941 (BF Goodrich) | 0.15 |
| | Deminaralised water | 25.00 |
| D | DM Water | 2.00 |
| | Triethanol amine | 0.10 |
| E | Tocopherol E acetate | 0.1 |
| F | Color | 0.15 |
| | Perfume | 0.5 |
| G | Ethanol | 2.0 |
| | Concentrate | 2.0 |

1. Heat Phase A to 75 degrees C. on water bath and melt the mixture
2. Disperse Phase B material and heat to 75 degree C. on water bath. Add Phase A to B under high speed stirring, mix well for 15 minutes,
3. Add phase C to Phase AB followed by Phase D, mix well for 15 minutes.
4. Add Phase E to Phase ABCD and stir for 5 minutes.
5. Add Phase F to Phase ABCDE and stir for 5 minutes.
6. Dissolve the active fraction in alcohol and add to Phase ABCDEF and stir for 5 minutes.

The above clearly reveal the possible forms of the cosmetic/dermopharmaceutical skin care formulation involving the concentrate or active ingredient. It is thus possible by way of the present invention to provide skin care agent in cosmetic/dermopharmaceutical compositions for topical application to human skin including leave on and wash-off products. The invention is further directed to cost-effective and natural extract based skin care for its wide benefit application and use as cosmetic/dermopharmaceutical formulations.

1. (canceled)

2. A skin care composition comprising selectively a synergistic combination of: (a) polygallate molecule of Formula 1:
(R′)₂₋₅—R″ Formula 1
wherein R′=galloyl, R″=polyhydroxy compounds including sugars; and
(b) a gallic acid ester of Formula 2 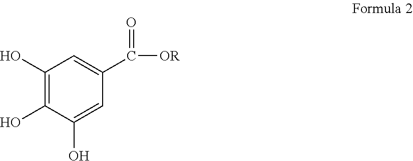
wherein R=an alkyl group in a ratio of (a):(b) of 80:20 to 40:60; as an anti-aging agent and any suitable skin care compatible carrier thereof.

3. A skin care composition according to claim 1, wherein the polygallate molecule is pentagalloyl glucose of Formula 3: 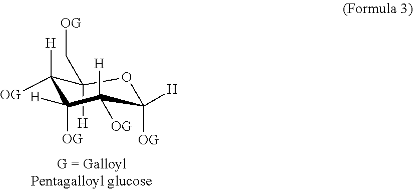
and gallic acid ester derivative comprises methyl gallate of Formula 4:

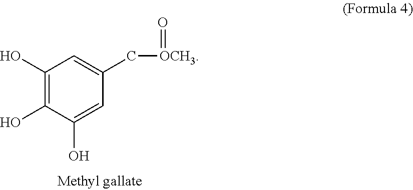

4. A skin care composition according to claim 1, comprising extracts from the skin/seed kernel of plant Mangifera indica Linn., containing mixture of Formula 1 and Formula 2 as an ingredient in the extract.

5-7. (canceled)

8. A skin care composition according to claim 1 for topical use comprising
a. about 0.0001 wt % to about 20 wt. % of said skin care agent based on the total weight of the ingredients in the composition; and
b. said any suitable skin care compatible carrier thereof.

9. A skin care composition according to claim 1 as a leave-on or a wash-off product in the form of creams, ointments, emulsions, gels, lotions, oils, sticks, sprays, soaps, packs, wraps, woven or nonwoven wipes, films or patches as a vehicle for topical application of the said skin care composition.

10-11. (canceled)

12. A process for the extraction of actives from Mangifera indica suitable for use as a skin benefiting composition especially anti aging composition comprising:
i) subjecting the air-dried fruit skin/kernel of Mangifera indica Linn., in powder form, to extraction with any organic solvent, preferably ethyl acetate/methanol, to provide an extract,
ii) concentrating the extract in vacuo to provide a concentrate,
iii) subjecting residue to column chromatography, preferably a silica gel column, using as eluents solvents such as ethyl acetate:chloroform (3:1), ethyl acetate, ethyl acetate:methanol (3:1) to provide fractions,
iv) subjecting the fractions to bioactivity screening in a elastase inhibition activity screening assay to provide active fractions,
v) pooling the active fractions and concentrating the resulting solution to dryness in vacuo to provide another concentrate,
vi) optionally, subjecting the concentrate to further chromatography and obtain more enriched concentrate; and
vii) subjecting the active fractions/enriched concentrate thus obtained to a process of crystallization to provide the desired skin benefit active.

13-14. (canceled)

15. A skin care composition according to claim 1 wherein compound (a) polygallate molecule of Formula 1
(R′)₂₋₅—R″ Formula 1
involves R′=galloyl, R″=sugars.
